(19) United States
(12) Patent Application Publication
(10) Pub. No.: US 2022/0387377 A1
(43) Pub. Date: Dec. 8, 2022

(54) (+)-CIS TETRAHYDROCANNABINOL ((+)-CIS-THC) FOR USE AS A MEDICAMENT

(71) Applicant: GW Research Limited, Cambridge (GB)

(72) Inventors: Geoffrey GUY, Cambridge (GB); Volker KNAPPERTZ, Cambridge (GB); Benjamin WHALLEY, Cambridge (GB); Marie WOOLLEY-ROBERTS, Cambridge (GB)

(21) Appl. No.: 17/771,190

(22) PCT Filed: Oct. 23, 2020
(86) PCT No.: PCT/GB2020/052679

§ 371 Date: Apr. 22, 2022

(30) Foreign Application Priority Data
Oct. 25, 2019 (GB) 1915514.2

(51) Int. Cl. A61K 31/352; A61P 25/04

(52) U.S. Cl. CPC A61K 31/352; A61P 25/04

(57) ABSTRACT

The present invention relates to a tetrahydrocannabinol (THC) type cannabinoid compound for use as a medicament. The THC-type cannabinoid is an enantiomer of the (−)-trans- tetrahydrocannabinol which is a naturally occurring cannabinoid that can be found in cannabis plant strains which have been bred to yield THC as the dominant cannabinoid. The particular enantiomer (+)-cis tetrahydrocannabinol has been found to have properties which are different from the naturally occurring (−)-trans-THC. The cannabinoid (+)-cis-THC has been found to occur in low concentrations in particular cannabis plant strains which have been bred to produce cannabidiol (CBD) as the dominant cannabinoid. Furthermore, the cannabinoid can be produced by synthetic means.

### FIELD OF THE INVENTION

The present invention relates to a tetrahydrocannabinol (THC) type cannabinoid compound for use as a medicament.

The THC-type cannabinoid is an enantiomer of the (−)-trans-tetrahydrocannabinol which is a naturally occurring cannabinoid that can be found in cannabis plant strains which have been bred to yield THC as the dominant cannabinoid. The particular enantiomer (+)-cis tetrahydrocannabinol has been found to have properties which are different from the naturally occurring (−)-trans-THC.

The cannabinoid (+)-cis-THC has been found to occur in low concentrations in particular cannabis plant strains which have been bred to produce cannabidiol (CBD) as the dominant cannabinoid. Furthermore, the cannabinoid can be produced by synthetic means.

Disclosed herein are data which demonstrate the efficacy of (+)-cis-THC in models of disease. In addition, a method for the synthesis of (+)-cis-THC is described.

### BACKGROUND TO THE INVENTION

Cannabinoids are natural and synthetic compounds structurally or pharmacologically related to the constituents of the cannabis plant or to the endogenous agonists (endocannabinoids) of the cannabinoid receptors CB1 or CB2. The only way in nature in which these compounds are produced is by the cannabis plant. Cannabis is a genus of flowering plants in the family Cannabaceae, comprising the species Cannabis sativa, Cannabis indica, and Cannabis ruderalis (sometimes considered as part of Cannabis sativa).

Cannabis plants comprise a highly complex mixture of compounds. At least 568 unique molecules have been identified. Among these compounds are cannabinoids, terpenoids, sugars, fatty acids, flavonoids, other hydrocarbons, nitrogenous compounds, and amino acids. With respect to the cannabinoids, over 100 different cannabinoids have been identified (see for example, Handbook of Cannabis, Roger Pertwee, Chapter 1, pages 3 to 15).

Cannabinoids exert their physiological effects through a variety of receptors including, but not limited to, adrenergic receptors, cannabinoid receptors (CB1 and CB2), GPR55, GPR3, or GPRS. The principle cannabinoids present in cannabis plants are the cannabinoid acids Δ9-tetrahydrocannabinolic acid (Δ9-THCA) and cannabidiolic acid (CBDA) with small amounts of their respective neutral (decarboxylated) cannabinoids. In addition, cannabis may contain lower levels of other minor cannabinoids. “Chemical composition, pharmacological profiling, and complete physiological effects of these medicinal plants, and more importantly the extracts from cannabis, remain to be fully understood.” Lewis, M. M. et al., ACS Omega, 2, 6091-6103 (2017).

The compound tetrahydrocannabinol (THC) in its natural form of (−)-trans-THC is psychoactive. Medical uses of (−)-trans-THC include its use to treat chemotherapy induced nausea and vomiting and in the treatment of HIV/AIDS related anorexia, (−)-trans-THC is also a component of nabiximols (Sativex) which is approved in Europe and Canada for the treatment approved for the spasticity associated with multiple sclerosis.

The tetrahydrocannabinol molecule is known to exist in four stereoisomers: (−)-trans-delta-9-tetrahydrocannabinol, (+)-trans-delta-9-tetrahydrocannabinol, (−)-cis-delta-9-tetrahydrocannabinol and (+)-cis-delta-9-tetrahydrocannabinol; see FIG. 1.

In synthetically produced drugs where there are chiral centres, and as such stereoisomers can be formed, it is important to understand the properties of the different enantiomers as oftentimes the synthesis forms a racemic mixture whereby both the (−) and (+) enantiomers are produced.

The pharmacological activity of THC is stereospecific; the (−)-trans-THC isomer (dronabinol) is 6-100 times more potent than the (+)-trans-THC isomer depending on the assay (Dewey et al., 1984).

In other medicaments both enantiomers have similar activities, for example both ibuprofen enantiomers have anti-inflammatory properties. Caution also needs to be taken to ensure that one of the enantiomers is not toxic or harmful to the patient.

In the case of THC which in addition to having optical or mirror image, (+) and (−) enantiomers, it also has geometric isomers which are termed cis and trans isomers. The FDA considers that due to the chemically distinct nature of geometric isomers these should be treated as separate drugs (https://www.fda.gov/regulatory-information/search-fda-guidance-documents/development-new-steroisometric-drugs).

The present invention demonstrates that surprisingly the compound (+)-cis-THC has been found to display therapeutic efficacy in animal models of disease. Heretofore this compound has not been found to have any therapeutic efficacy.

### BRIEF SUMMARY OF THE DISCLOSURE

In accordance with a first aspect of the present invention there is provided (+)-cis tetrahydrocannabinol ((+)-cis-THC) for use as a medicament.

Preferably the (+)-cis-THC is in the form of a plant extract. More preferably the (+)-cis-THC is in the form of a highly purified extract of cannabis.

Preferably the highly purified extract comprises at least 80% (w/w) (+)-cis-THC, more preferably the highly purified extract comprises at least 85% (w/w) (+)-cis-THC, more preferably the highly purified extract comprises at least 90% (w/w), more preferably the highly purified extract comprises at least 95% (w/w) (+)-cis-THC, more preferably still the highly purified extract comprises at least 98% (w/w) (+)-cis-THC.

Alternatively, the (+)-cis-THC is present as a synthetic compound.

Preferably the dose of (+)-cis-THC is greater than 100 mg/kg/day. More preferably the dose of (+)-cis-THC is greater than 250 mg/kg/day. More preferably the dose of (+)-cis-THC is greater than 500 mg/kg/day. More preferably the dose of (+)-cis-THC is greater than 750 mg/kg/day. More preferably the dose of (+)-cis-THC is greater than 1000 mg/kg/day. More preferably the dose of (+)-cis-THC is greater than 1500 mg/kg/day.

Alternatively, the dose of (+)-cis-THC is less than 100 mg/kg/day. More preferably the dose of (+)-cis-THC is less than 50 mg/kg/day. More preferably the dose of (+)-cis-THC is less than 20 mg/kg/day. More preferably the dose of (+)-cis-THC is less than 10 mg/kg/day. More preferably the dose of (+)-cis-THC is less than 5 mg/kg/day. More preferably the dose of (+)-cis-THC is less than 1 mg/kg/day. More preferably the dose of (+)-cis-THC is less than 0.5 mg/kg/day.

In accordance with a second aspect of the present invention there is provided a composition for use as a medicament comprising (+)-cis tetrahydrocannabinol ((+)-cis-THC), and one or more pharmaceutically acceptable excipients.

In accordance with a third aspect of the present invention there is provided a (+)-cis tetrahydrocannabinol ((+)-cis-THC) for use in the treatment of epilepsy.

In accordance with a fourth aspect of the present invention there is provided a method for the production of (+)-cis tetrahydrocannabinol ((+)-cis-THC).

### BRIEF DESCRIPTION OF THE DRAWINGS

Embodiments of the invention are further described hereinafter with reference to the accompanying drawings, in which:

FIG. 1 shows the four stereoisomers of tetrahydrocannabinol;

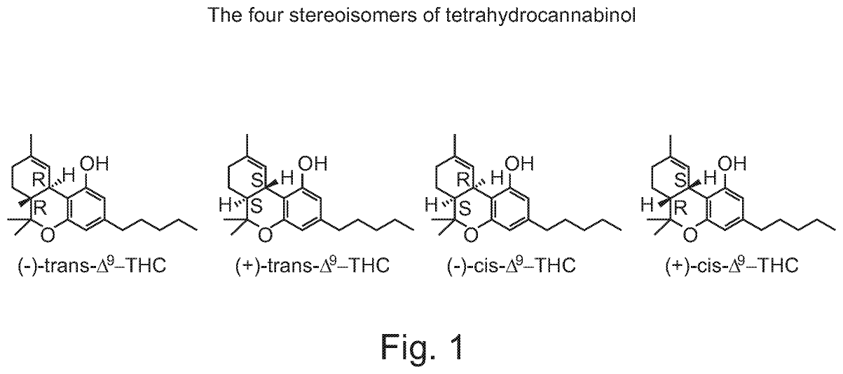

FIG. 2 shows a superimposition at the phenolic ring level of the optimized conformations of (−)-trans-Δ9-THC (magenta), (+)-cis-Δ9-THC (dark pink) and (−)-cis-Δ9-THC (light pink) and (+)-cis-Δ9-THC (orchid) in stick representation;

FIG. 3 shows representative frames from MD simulation of CB1R in complex with (−)-trans-THC (Panel A), (−)-cis-THC (Panel B) and (+)-cis-THC (Panel C);

FIG. 4 shows representative frames from MD simulation of CB2R in complex with (−)-trans-THC (Panel A), (−)-cis-THC (Panel B) and (+)-cis-THC (Panel C); and

FIG. 5 shows a HPLC chromatogram showing the separation of the cis enantiomers on a semi-preparative column.

### DEFINITIONS

“Cannabinoids” are a group of compounds including the endocannabinoids, the phytocannabinoids and those which are neither endocannabinoids or phytocannabinoids, hereinafter “syntho-cannabinoids”.

“Endocannabinoids” are endogenous cannabinoids, which are high affinity ligands of CB1 and CB2 receptors.

“Phytocannabinoids” are cannabinoids that originate in nature and can be found in the cannabis plant. The phytocannabinoids can be present in an extract including a botanical drug substance, isolated, or reproduced synthetically.

“Syntho-cannabinoids” are those compounds that are not found endogenously or in the cannabis plant. Examples include WIN 55212 and rimonabant.

An “isolated phytocannabinoid” is one which has been extracted from the cannabis plant and purified to such an extent that all the additional components such as secondary and minor cannabinoids and the non-cannabinoid fraction have been removed.

A “synthetic cannabinoid” is one which has been produced by chemical synthesis. This term includes modifying an isolated phytocannabinoid, by, for example, forming a pharmaceutically acceptable salt thereof.

A “substantially pure” cannabinoid is defined as a cannabinoid which is present at greater than 95% (w/w) pure. More preferably greater than 96% (w/w) through 97% (w/w) thorough 98% (w/w) to 99% % (w/w) and greater.

“Stereoisomers” are molecules that are identical in atomic constitution and bonding but differ in the three-dimensional arrangement of the atoms.

“Geometric isomers” are chemically distinct and pharmacologically different enantiomers and are generally readily separated without chiral techniques.

“Diastereoisomers” are isomers of drugs with more than one chiral centre that are not mirror images of one another.

### DETAILED DESCRIPTION

The present invention provides data to demonstrate the different physicochemical properties of the claimed compound, (+)-cis-tetrahydrocannabinol versus its optical and geometric isomers. Furthermore, data is presented to demonstrate the efficacy of this compound in an animal model of disease. As an additional aspect there is provided a method for the synthetic production of (−)-cis-THC).

### EXAMPLE 1: IN SILICO VIRTUAL SCREENING FOR ISOMERS OF PHYTOCANNABINOIDS

Using a combined approach of molecular docking and molecular dynamics in membrane environment allowed the identification of the putative binding modes of (+)-cis-THC to the CB1 and CB2 cannabinoid receptors in comparison to the other stereoisomers of THC.

### Methods

### Computational Methods:

Starting ligand geometries were built with Chemical 2.99.23, followed by energy minimization (EM) at molecular mechanics level first, using Tripos 5.2 force field parametrization, and then at AM1 semi-empirical level; fully optimized using GAMESS program4 at the Hartree-Fock level with STO-3G basis set; subjected to HF/6-31G*/STO-3G single-point calculations to derive the partial atomic charges by the RESP procedure5.

Docking studies were performed with AutoDock 4.2 distribution, by using the crystallographic structures of the CB1R complexed to the agonist AM11542 (PDB id:5XRA) and CB2R complexed to the antagonist AM10257 (PDB id:5ZTY).

Both proteins and ligands were processed with AutoDock Tools (ADT) package version 1.5.6rc16 to merge nonpolar hydrogens, calculate Gasteiger charges and select the rotatable side-chain bonds.

Grids for docking evaluation with a spacing of 0.375 Å and 60×70×60 points, centered on the ligand binding site, were generated using the program AutoGrid 4.2 included in Autodock 4.2 distribution.

Different runs were carried out by using different combinations of flexible residues.

Lamarckian Genetic Algorithm (LGA) was adopted to perform molecular docking along with the following docking parameters: 100 individuals in a population with a maximum of 15 million energy evaluations and a maximum of 37000 generations, followed by 300 iterations of Solis and Wets local search. A total of 100 docking runs were performed for each calculation.

The loops missing in the crystallographic structures used in this study, were modelled with MODELLER v9.11 program7.

Representative complexes for each combination of ligand and receptor were completed by addition of all hydrogen atoms and underwent energy minimisation. The energy minimized complexes were embedded in POPC bilayer using CHARMM-GUI web-interface and then molecular dynamics (MD) simulations in membrane environment were carried out with pmemd.cuda module of Amber16 package8, using lipid 14ff for lipids, ff14SB force field for the protein and gaff parameters for the ligands. MD production runs were carried out for 100 ns.

### Results

### Comparison of the THC Isomers:

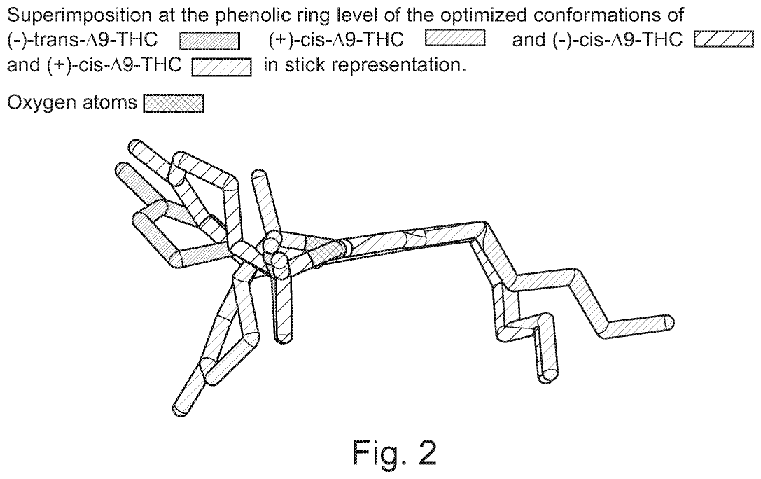

To compare the conformation of the polycyclic moieties, the 3D coordinates of the THC isomers, obtained as described in methods section, were superimposed at level of the phenolic ring and shown in FIG. 2.

It was found that the (−)-cis-THC isomer fits onto the scaffold of the (−)-trans-THC at level of the dimethyl-pyran moiety, with the tetrahydrobenzomethyl rings pointing in the same direction.

However, the conformation of the (+)-cis THC isomer largely differs from both (−)-trans and (−)-cis isomer.

Theoretical Binding at the CB1 Receptor (CB1R):

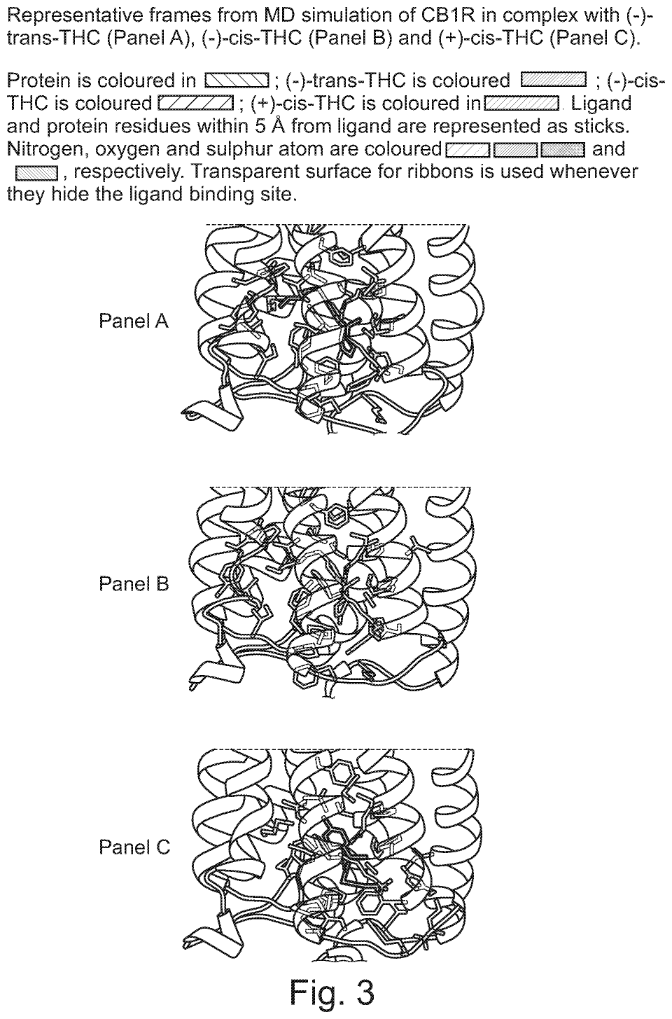

The x-ray structure of the agonist-bound CB1R was selected for docking studies as (−)-trans-THC is a known CB1R partial agonist as shown in FIG. 3A.

The cis isomers of THC were docked in the same x-ray structure for comparative purposes. FIG. 3B demonstrates the docking of the (−)-cis-THC and FIG. 3C demonstrates the docking of the (+)-cis-THC.

As can be seen, both (−)-trans-THC and (−)-cis-THC adopted an L-shaped conformation. The pentyl chain is pointing toward Trp2795.43 on helix V, tricyclic ring system forming π˜π and hydrophobic interactions with Phe268 on the loop ECL2, Phe3797.35, Phe1893.25 and Phe1772.64 and a hydrogen bond with Ser3837.39.

The pentyl chain also engages hydrophobic interaction with Phe2003.36, a key residue in CB1R activation because it is part of the toggle switch with Trp3566.48. In fact, the π—π stacking between Trp3566.48 and Phe2003.36 stabilizes the inactive form of the receptor.

The (−)-cis-THC adopts the same pose of (−)-trans-THC, with the exception of the tetrahydro-methyl-benzene group which is tilted in comparison to that of trans-THC.

However, the (+)-cis-THC adopts a reversed orientation in the tricyclic ring, with the pentyl chain pointing toward the N-terminus and Phe1772.64, far from Phe2003.36 (FIG. 3C).

### Theoretical Binding at the CB2 Receptor:

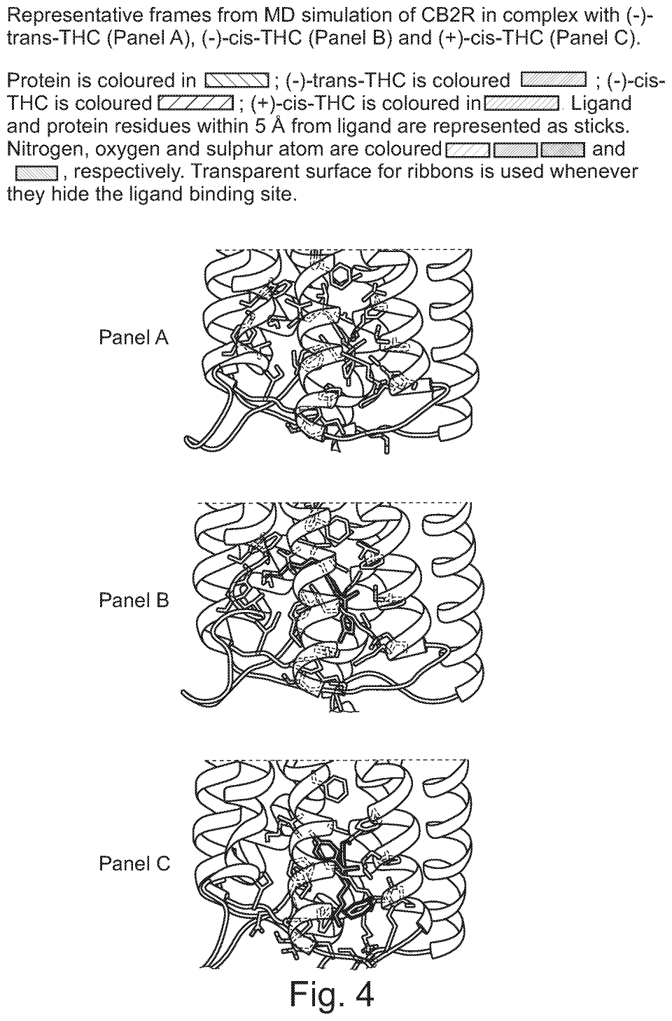

The binding of (−)-trans-THC to the CB2R is shown in FIG. 4A.

The cis isomers of THC were docked in the same x-ray structure for comparative purposes. FIG. 4B demonstrates the docking of the (−)-cis-THC and FIG. 4C demonstrates the docking of the (+)-cis-THC.

The overall arrangement of the investigated compounds within CB2R ligand binding site well overlaps that already observed in CB1R complexes. The interactions between (−)-trans-THC and CB2 are mainly hydrophobic and aromatic and involves residues from ECL2 as well as helices II, Ill, V, and VI. The tricyclic ring of THC forms interactions with Phe183ECL2 and hydrophobic interactions with Phe1063.25 and Phe942.64, while the pentyl chain forms hydrophobic interactions with Trp1945.43 and Phe1173.36. This latter residue is part of the switch toggle along with Trp2586.48. The hydroxy group of the tricyclic terpenoid ring of THC engages an H-bond with Ser2857.39.

Similar to the binding at CB1R, the (−)-cis-THC isomer adopts a similar orientation to (−)-trans-THC, whereas the (+)-cis-THC is reversed in the ligand binding site.

### Conclusions

The combined approach of molecular docking and molecular dynamics allowed the determination of the putative binding modes of both (−)-trans-THC and (−)-cis-THC and (+)-cis-THC isomers within the ligand binding site of CB1 and CB2 receptors.

The binding modes of the three compounds into the two receptors are similar since both the residues and the overall arrangements of helices, N-termini and ECL2 loops are well conserved between the two subtypes.

The two cis isomers of THC differ greatly from each other in the conformation of the tricyclic scaffold, with the (−)-cis-THC being more similar to (−)-trans-THC. The (+)-cis-THC adopts a reversed binding mode within the ligand binding site of both receptors and a different functional profile is expected for this isomer.

### EXAMPLE 2: EVALUATION OF THE ANTICONVULSANT EFFECT OF (+)-CIS-THC IN THE MOUSE SUPRAMAXIMAL ELECTROSHOCK SEIZURE (MES) MODEL OF GENERALIZED SEIZURES

The efficacy of (+)-cis-THC was tested in a mouse model of seizure, the maximal electroshock (MES) test.

### Methods

Mice were administered MES (30 mA, rectangular current: 0.6 ms pulse width, 0.2 s duration, 50 Hz) via corneal electrodes connected to a constant current shock generator (Ugo Basile: type 7801) to reliably produce tonic hind limb convulsions. The number of tonic convulsions was recorded.

Sixteen mice were studied per group. The test was performed blind.

The test substance, (+)-cis-THC, was evaluated at 4 doses (10, 50, 100 and 150 mg/kg), administered i.p. 60 minutes before MES, and compared with a vehicle control group (administered under the same experimental conditions).

Valproate (positive control) was administered at 250 mg/kg i.p. 30 minutes before MES, was used as a reference substance and was compared with a vehicle group (administered i.p. 60 minutes before MES).

Data was analysed by comparing treated groups with the appropriate vehicle control using 2-tailed Fisher's Exact Probability tests (p<0.05 considered significant).

### Results

Table 1 demonstrates the data produced in this experiment.

In the positive control (valproate) group there was a significant 100% change in the number of tonic clonic seizures observed in the animals compared to vehicle, demonstrating an expected anti-convulsant effect.

In the (+)-cis-THC treated mice, a dose-related increase in the percentage change in the number of tonic clonic seizures observed in the animals compared to vehicle was observed.

At the highest dose of 150 mg/kg, administered i.p. 60 minutes before the test, this resulted in a significant (p<0.05), 37.5% reduction, in tonic convulsions compared with vehicle control.

No effect was observed at the lowest dose (10 mg/kg).

TABLE 1
Percentage change in number of tonic clonic
seizures compared to vehicle after MES

| | | | | | % change | |
|---|---|---|---|---|---|---|
| | Dose | | \hat{ }PTT | | from | |
| Treatment | (mg/kg) | Route | (mins) | N | vehicle | Significance |
| Vehicle | 10 | I.P. | 60 | 16 | — | — |
| Valproate | 250 | I.P. | 30 | 16 | 100.0 | *** |
| (+)-cis-THC | 10 | I.P. | 60 | 16 | 0.0 | ns |
| (+)-cis-THC | 50 | I.P. | 60 | 16 | 18.8 | ns |
| (+)-cis-THC | 100 | I.P. | 60 | 16 | 25.0 | ns |
| (+)-cis-THC | 150 | I.P. | 60 | 16 | 37.5 | * |

\hat{ }PTT (Pre-treatment time). % change from vehicle refers to the anticonvulsant effect by the treatment compared to vehicle.
* p < 0.05 and *** p < 0.001 significant inhibition of tonic hindlimb seizures when compared to the corresponding vehicle control (Fisher's test).
Ns: non significantly different from vehicle control (Fisher's test).

### Conclusions

These data demonstrate that the enantiomer (+)-cis-THC produced a significant anti-convulsant effect in the MES model. These data are the first data to demonstrate a therapeutic effect of this enantiomer of THC.

### EXAMPLE 3: ASSESSMENT OF ANTI-NOCICEPTIVE POTENTIAL OF (+)-CIS-THC IN THE MOUSE USING HOTPLATE METHOD

The efficacy of (+)-cis-THC was tested in a mouse model of pain, the hotplate test.

### Methods

Mice underwent a minimum habituation period of 7 days prior to study commencement. Naïve mice were acclimatised to the procedure room in their home cages, with food and water available ad libitum.

Animals were treated with either treatment vehicle, (+)-cis-THC at 1,15, 50,100, 125 and 150 mg/kg at 10 ml/kg i.p. or Morphine 10 mg/kg or Morphine vehicle (Saline) at 10 ml/kg i.p.

Animals were placed onto a hot plate set at 52° C. and time to withdrawal threshold (first response of lifting, licking front or hind paws or trying to escape) was taken at 1-hour post treatment, or at 0.5 hour after the positive control.

Animals were culled by a schedule 1 method, immediately after the measurement.

Data were analysed by comparing withdrawal thresholds back to Vehicle treated group

### Results

Table 2 demonstrates the data produced in this experiment.

In the positive control (morphine) group there was a significant increase in the withdrawal threshold compared to vehicle, demonstrating an expected anti-nociceptive effect.

In the (+)-cis-THC treated mice, none of the doses tested produced a significant difference when compared to vehicle.

TABLE 2
Withdrawal threshold of animals after treatment

| | | | | | Withdrawal |
|---|---|---|---|---|---|
| | | Dose | | | threshold (sec) |
| Groups | Treatment | mg/kg | Route | N | Mean +/− SEM |
| 1 | Vehicle | — | I.P. | 12 | 11.5 +/− 0.96 |
| 2 | (+)-cis-THC | 1 | I.P. | 12 | 10.23 +/− 0.64 |
| 3 | (+)-cis-THC | 15 | I.P. | 12 | 9.96 +/− 0.73 |
| 4 | (+)-cis-THC | 50 | I.P. | 12 | 9.80 +/− 0.80 |
| 5 | (+)-cis-THC | 100 | I.P. | 12 | 10.67 +/− 0.77 |
| 6 | (+)-cis-THC | 125 | I.P. | 12 | 11.18 +/− 1.18 |
| 7 | (+)-cis-THC | 150 | I.P. | 12 | 11.68 +/− 0.52 |
| 8 | Vehicle | — | I.P. | 12 | 10.23 +/− 0.59 |
| 9 | Morphine | 10 | I.P. | 12 | 17.48 +/− 1.08 *** |

*** p < 0.001 significant when compared to the corresponding vehicle control (Fisher's test).

### Conclusions

These data demonstrate that the enantiomer (+)-cis-THC has no anti-nociceptive effect in an animal model of pain.

### EXAMPLE 4: SYNTHETIC PRODUCTION METHOD FOR (+)-CIS-TETRAHYDROCANNABINOL

As previously described the compound (+)-cis-THC is produced as a minor cannabinoid by cannabis plants which predominantly produces the cannabinoid cannabidiol (CBD). In a highly purified extract of CBD the amount of (+)-cis-THC which remains in the extract very small given that the amount of total THC is approximately less than 0.1% (w/w) based on total amount of cannabinoid in the preparation.

It is known that the THC in the purified extract produced from a CBD producing cannabis plant is present as both geometric isomers of trans and cis THC. We also know that the ratio of the trans-THC:cis-THC changes during processing and purification of the extract from about 3.6:1 trans-THC:cis-THC to about 0.8:1 trans-THC:cis-THC.

It has further been discovered that the cis-THC present in the purified preparation is present as a mixture of the optical isomers (−)-cis-THC and (+)-cis THC. The ratio of the (−)-cis-THC:(+)-cis-THC is in the range of approximately 9:1 ((−)-cis-THC:(+)-cis-THC).

Given the very low levels of the compound (+)-cis-THC found in nature a synthetic pathway, described below as Scheme 1, details a methodology that can be used in order to produce the cannabinoid (+)-cis-THC in larger quantities.

The compounds are numbered, and their full names provided in the box below the pathway.

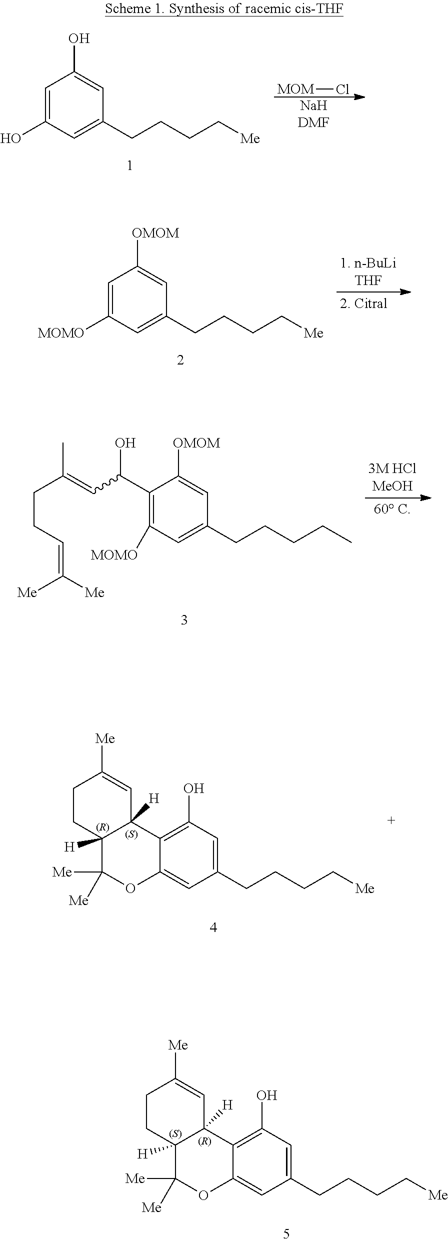

| Compound | Name |
|---|---|
| 1 | Olivetol |
| 2 | bis-MOM-protected olivetol |
| 3 | the alcohol intermediate of bis-MOM-protected olivetol |
| 4 | (+)-cis-THC |
| 5 | (−)-cis-THC |
| MOM-Cl | Methoxymethyl chloride |

The resulting racemate of cis-THC was separated using chiral separation of enantiomers using HPLC column Phenomenex Lux Cellulose 2 chiral column.

A reversed-phase gradient of MeCN/H2O(0.1% HCO2H) was used.

Each material isolated was analysed by optical rotation, chiral HPLC, LCMS and 1H NMR.

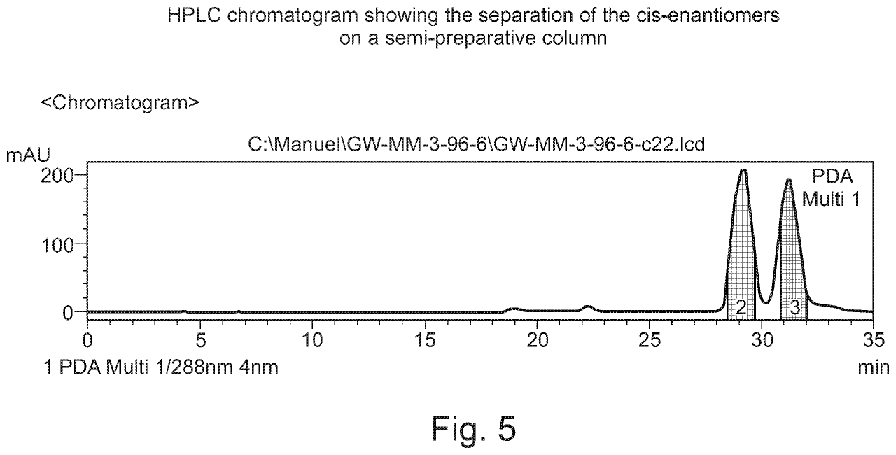

FIG. 5 shows an HPLC chromatogram where Peak 2 was observed to be (+)-cis-THC with an enantiomeric excess of 99.4%, while peak 3 was identified as (−)-cis-THC with an enantiomeric excess of 96.8%.

1. (+)-cis tetrahydrocannabinol ((+)-cis-THC) for use as a medicament.

2. (+)-cis-THC for use according to claim 1, wherein the (+)-cis-THC is in the form of a plant extract.

3. (+)-cis-THC for use according to claim 2, wherein the (+)-cis-THC is in the form of a highly purified plant extract.

4. (+)-cis-THC for use according to claim 3, wherein the (+)-cis-THC comprises at least 80% (w/w) (+)-cis-THC.

5. (+)-cis-THC for use according to claim 3, wherein the (+)-cis-THC comprises at least 95% (w/w) (+)-cis-THC.

6. (+)-cis-THC for use according to claim 1, wherein the (+)-cis-THC is in the form of a synthetic compound.

7. (+)-cis-THC for use according to any of the preceding claims, wherein the dose of (+)-cis-THC is greater than 100 mg/kg/day.

8. (+)-cis-THC for use according to any of the preceding claims, wherein the dose of (+)-cis-THC is less than 100 mg/kg/day.

9. A composition for use as a medicament comprising (+)-cis tetrahydrocannabinol ((+)-cis-THC) and one or more pharmaceutically acceptable excipients.

10. (+)-cis tetrahydrocannabinol ((+)-cis-THC) for use in the treatment of pain.

11. A process for the preparation of (+)-cis tetrahydrocannabinol ((+)-cis-THC).
